(19) United States
(12) Patent Application Publication
(10) Pub. No.: US 2024/0376518 A1
(43) Pub. Date: Nov. 14, 2024

(54) METHOD FOR DETECTING TARGET NUCLEIC ACIDS USING PLASMONIC IMMUNOMAGNETIC NANOPARTICLES

(71) Applicant: Soonchunhyang University Industry Academy Cooperation Foundation, Asan-si (KR)

(72) Inventor: Jung-Hoon LEE, Seoul (KR)

(73) Assignee: Soonchunhyang University Industry Academy Cooperation Foundation, Asan-si (KR)

(21) Appl. No.: 18/584,279

(22) Filed: Feb. 22, 2024

(30) Foreign Application Priority Data
May 9, 2023 (KR) 10-2023-0059804

(51) Int. Cl. C12Q 1/6804; C12N 15/10; C12Q 1/6844

(52) U.S. Cl. CPC C12Q 1/6804; C12N 15/1003; C12Q 1/6844

(57) ABSTRACT

One aspect of the present invention relates to a method for detecting target nucleic acids using plasmonic immunomagnetic nanoparticles. The method for detecting target nucleic acids using plasmonic immunomagnetic nanoparticles according to one aspect of the present invention is low-cost, allows quick and simple detection of target nucleic acids, and thus can be used in various fields such as molecular biology, medicine, and biological classification.

### STATEMENT REGARDING PRIOR DISCLOSURES BY THE INVENTOR OR A JOINT INVENTOR

Applicant designates the following article as a grace period publication in order to expedite examination of the application in accordance with 37 CFR 1.77(b)(6) and MPEP 608.01(a): “Ultrasensitive Plasmonic Photothermal Immunomagnetic Bioassay Using Real-Time and End-Point Dual-Readout” published in Sensors and Actuators B: Chemical, Volume 377, on Feb. 15, 2023. The disclosures of the article are incorporated herein by reference in their entirety for all purposes.

### CROSS-REFERENCE TO RELATED APPLICATION

This application claims priority to and the benefit of Korean Patent Application No. 10-2023-0059804, filed on May 9, 2023, the disclosure of which is incorporated herein by reference in its entirety.

### SEQUENCE LISTING

The instant application contains a Sequence Listing which has been submitted electronically in XML format and is hereby incorporated by reference in its entirety. Said XML copy, created on Mar. 11, 2024 is named “4669-220.xml” and is 2,982 bytes in size.

### TECHNICAL FIELD

The present invention relates to a method for detecting target nucleic acids using plasmonic immunomagnetic nanoparticles.

This application was supported by the Ministry of Science and ICT (No. NRF-2022R1F1A1070162 (1711171631, 1711186785) and No. 2023-DD-UP-0007) and the Ministry of Education (No. NRF-2021R1A6A1A03039503 (1345362911)).

### BACKGROUND OF THE INVENTION

Immuno-polymerase chain reaction (iPCR) is an immunoassay for ultra-sensitive quantification of proteins using the specificity of antigen-antibody interaction and the amplification ability of PCR. In general, iPCR can increase sensitivity by thousands to hundreds of millions of times compared to ELISA (enzyme-linked immunosorbent assay) due to the exponential amplification ability of PCR. However, existing qPCR thermal cyclers used in iPCR are sophisticated but expensive, time- and energy-consuming Peltier-based thermal block modules, so their application to point of care (POC) diagnosis is limited.

Recently, photonic PCR using the photothermal effect of plasmonic nanomaterials is attracting a lot of attention due to its fast and high photothermal conversion capability. However, photonic PCR still has some problems, limiting its widespread PCR application.

Accordingly, in order to solve the above problems, the present inventors developed plasmonic photothermal quantitative iPCR (PPT-qiPCR) using multifunctional plasmonic immunomagnetic nanoparticles (PIMN).

### SUMMARY OF THE INVENTION

### Technical Problem

One aspect of the present invention is directed to providing a method for detecting target nucleic acids, including (a) binding capture antibody and plasmonic immunomagnetic nanoparticles (PIMNs); (b) binding detection antibody and streptavidin-nucleic acid complex; (c) mixing the nanoparticles bound to the capture antibody and the complex bound to the detection antibody; (d) separating the mixed nanoparticles and complex; (e) amplifying target nucleic acids using the complex, primer, and nucleic acid polymerase; and (f) detecting target nucleic acids.

Another aspect of the present invention is directed to providing multifunctional plasmonic immunomagnetic nanoparticles, comprising iron oxide nanoclusters, gold nanoshells and antibodies.

### Technical Solution

One aspect of the present invention provides a method for detecting target nucleic acids, including (a) binding capture antibody and plasmonic immunomagnetic nanoparticles (PIMNs); (b) binding detection antibody and streptavidin-nucleic acid complex; (c) mixing the nanoparticles bound to the capture antibody and the complex bound to the detection antibody; (d) separating the mixed nanoparticles and complex; (e) amplifying target nucleic acids using the complex, primer, and nucleic acid polymerase; and (f) detecting target nucleic acids.

The term “streptavidin” refers to a 52 kDa protein purified from Streptomyces avidinii.

The term “primer” refers to a short genetic sequence that serves as the starting point for creating another polymer strand complementary to the template during DNA synthesis.

In an embodiment, in step (a), the plasmonic immunomagnetic nanoparticles may have magnetism and photothermal properties.

The term “magnetism” means that a magnet has some influence on surrounding objects.

The term “photothermal properties” refers to properties that can specifically stimulate a desired area using light energy.

The term “plasmonic immunomagnetic nanoparticles (PIMNs)” refers to nanoparticles composed of iron oxide nanoclusters, gold nanoshells, and antibodies with magnetism and photothermal properties.

In an embodiment, in step (a), the capture antibody may be covalently bound to the surface of the nanoparticle.

The term “capture antibody” refers to an antibody that can specifically bind to one or more target proteins (antigens).

The term “covalent bond” refers to a bond created when atoms share electrons during a chemical bond.

In an embodiment, in step (b), the detection antibody may be biotinylated.

The term “detection antibody” refers to an antibody that can bind to a target protein (antigen) captured by the capture antibody.

In an embodiment, the detection efficiency may be increased by biotinylating the detection antibody.

In an embodiment, in step (c), the nanoparticles and the complex may be mixed in a sandwich structure.

In an embodiment, in step (d), the nanoparticles and the complex may be magnetically separated.

In an embodiment, in step (e), the polymerase may be at least one selected from the group consisting of Taq polymerase, VENT polymerase, DEEPVENT polymerase, PWO polymerase, and Pfu polymerase.

In an embodiment, in step (e), the amplification may be performed through photothermal cyclic amplification.

In an embodiment, in step (f), the detection may be performed through at least one selected from the group consisting of colorimetric assay, fluorescence assay, Raman assay, and gel electrophoresis.

The term “colorimetric assay” refers to testing or quantifying the concentration of a chemical compound or solution by measuring the absorbance of a specific wavelength of light using color reagents or the like.

The term “fluorescence assay” is a chemical analysis method using the fluorescence of a substance, which means converting a non-fluorescent sample into a fluorescent substance through a chemical reaction and analyzing the fluorescence.

The term “Raman assay” refers to generating (exciting) molecular motion using light, and analyzing these interactions to chemically analyze the sample.

The term “gel electrophoresis” refers to separating DNA, RNA, proteins, etc. by flowing an electric current through a gel matrix.

Another aspect of the present invention provides multifunctional plasmonic immunomagnetic nanoparticles, comprising iron oxide nanoclusters, gold nanoshells and antibodies.

The “iron oxide nanoclusters”, “gold nanoshells”, “antibodies”, “plasmonic immunomagnetic nanoparticles”, etc. may be within the above-mentioned range.

In an embodiment, the iron oxide nanoclusters may have magnetism.

In an embodiment, the gold nanoshells may have photothermal properties.

### Advantageous Effects

The method for detecting target nucleic acids using plasmonic immunomagnetic nanoparticles according to one aspect of the present invention is low-cost, allows quick and simple detection of target substances, and thus can be used in various fields such as molecular biology, medicine, and biological classification.

### BRIEF DESCRIPTION OF THE DRAWINGS

FIG. 1 is a schematic diagram of a workflow of plasmonic photothermal quantitative immuno-PCR using plasmonic immunomagnetic nanoparticles for IL-6 detection.

FIGS. 2A to 2F are diagrams showing results of confirming the characteristics of PIMN. Specifically, FIG. 2A is a diagram showing a result of confirming transmission electron microscopy (TEM), high-angle annular dark-field and elemental mapping images of PMNs (Au: red, Fe: green), and magnetic nanocluster cores. FIGS. 2B to 2D are diagrams showing results of confirming hydrodynamic diameter distribution (B), zeta potential (C) and UV-Vis spectrum (D) of PMNs at different surface modification steps, respectively. FIG. 2E is a diagram showing a result of confirming temperature profile of one photothermocycle between 72 and 95° C. with varying optical densities of PIMNs. FIG. 2F is a diagram showing a result of confirming calculated heating and cooling rates.

FIGS. 3A to 3D are diagrams showing images of iron oxide (A), (B) Fe3O4@Au seeds, (C) Fe3O4@Au, and (D) Fe3O4@Au-MPA confirmed through scanning electron microscopy (scale bar: 500 nm).

FIG. 4 is a diagram showing an EDX pattern (A) of Fe₃O₄@Au and an XRD pattern (B) of Fe₃O₄ and Fe₃O₄@Au.

FIG. 5 is a diagram showing a home-build apparatus for PPT-qiPCR.

FIG. 6 is a diagram showing a result of confirming a temperature profile of a solution without PIMNs.

FIG. 7 is a diagram showing a result of confirming thermal stability before and after 35 photothermocycles.

FIG. 8 is a diagram showing a result of confirming amplification curves of real-time PPT-qiPCR with target IL-6 (1000 pg/ml) and different amount SG dye (panel A), 3% agarose gel electrophoresis assay (panel B) and fluorescence spectrum (SG7×).

FIG. 9 is a diagram showing a result of confirming amplification curves of real-time PPT-qiPCR obtained from IL-6 (1000 pg/mL) and different concentrations of PIMNs.

FIGS. 10A to 10G are diagrams showing results of confirming characteristics of PPT-qiPCR using PIMNs. Specifically, FIG. 10A is a schematic diagram of PPT-qiPCR using real-time and end-point fluorescence dual-readout. FIG. 10B is a diagram showing a result of confirming a representative photothermal amplification temperature profile. FIG. 10C is a diagram showing a result of confirming real-time amplification curves of PPT-qiPCR at different concentrations of IL-6 (1000, 100, 50, 10, 5, 1 pg·mL⁻¹). FIG. 10D is a diagram showing a result of confirming Cq values from real-time amplification curves. FIG. 10E is a diagram showing a result of confirming end-point fluorescence curves of PPT-qiPCR from same samples in FIG. 10C (Red and black plots showed the end-point fluorescence signals before and after the magnetic separation, respectively). FIG. 10F is fluorescence and gel electrophoresis images of PPT-qiPCR (lane 1-9: 0, 0.25, 0.05, 0.5, 1, 10, 100, 500, 1000 pg·mL⁻¹). FIG. 10G is a diagram showing a result of selectivity test of PPT-qiPCR [Phosphate Buffered Saline (PBS) buffer, tumor necrosis factor-α (TNF-α), hemoglobin (Hb)].

FIG. 11 is a diagram showing the fluorescence quenching effect by fixed concentration of target and nanoparticles with different concentrations.

FIGS. 12A to 12C are diagrams showing results of performing colorimetric PPT-qiPCR for detection of IL-6. Specifically, FIG. 12A is a schematic diagram of workflow of end-point colorimetric PPT-qiPCR. FIG. 12B is an image of a result of performing end-point colorimetric PPT-qiPCR. FIG. 12C is a diagram showing a result of confirming the absorbance plot of the solution of end-point colorimetric PPT-qiPCR at 650 nm.

FIG. 13 is a diagram showing results of (A) pH changes of iPCR buffer over addition of MES buffer and (B) comparison of the measured absorbance after 35 thermocycling depending on the amount of TMB concentration.

FIG. 14 is a diagram showing a result of confirming optimization of irradiation time.

### DETAILED DESCRIPTION OF THE EMBODIMENTS

Hereinafter, the present invention will be described in more detail through examples. However, these examples are for illustrative purposes only and the scope of the present invention is not limited to these examples.

### Examples

### 1. Experimental Materials and Methods

### (1) Experimental Materials

Iron chloride ferric hexahydrate (FeCl₃·6H₂O), 3-aminopropyltrimethoxysilane (APTMS), tetrakis(hydroxymethyl)phosphonium chloride (THPC), hydrogen tetrachloroaurate trihydrate (HAuCl₄·3H₂O), sodium acetate, trisodium citrate dehydrate, potassium carbonate (K₂CO₃), and bovine serum albumin (BSA) were purchased from Sigma-Aldrich. Formaldehyde (HCHO) was obtained from TCI Company, and 3,3′, 5,5′-tetramethylbenzidine (TMB) and 3-Mercaptopropionic acid (MPA) were purchased from Energy Chemical. Purified anti-human IL-6 antibody (clone MQ2-13A5, rat monoclonal), biotin anti-human IL-6 antibody (clone MQ2-39C3, rat monoclonal), recombinant human IL-6, and purified streptavidin were purchased from BioLegend. Forward primer (5′-CATCGTCTGCCTGTCATGGGCTGTTAAT-3′: SEQ ID NO: 1), reverse primer (5′-TCGCCAGCTTCAGTTCTCTGGCATTT-3′: SEQ ID NO: 2), and a 112-base pair (bp) X-DNA target with a biotin label at the 5′ end were purchased from Integrated DNA Technologies (Coralville, IA, USA). Dream Taq DNA polymerase (0.05 U·μL⁻¹), reaction buffer (MgCl₂, 4 mM), dNTPs mixture (0.4 mM each) and MES buffer (8 μL, BupH™ MES buffered saline packs, 28390, 0.1 M MES, 0.9% sodium chloride, pH 4.7) were purchased from Thermo Fisher Scientific, and SYBR® Green I (excitation at 497 nm and emission at 530 nm) were purchased from Molecular Probes, Inc. Quick-Load® purple low molecular weight DNA ladder, Quick-Load® purple 1 kb DNA ladder and gel loading dye (purple, 6×) were purchased from New England BioLabs, Inc. Morphology and size distribution of PMNs and other intermediates were examined with a transmission electron microscope (TEM, Tecnai 12, Philips) and Field Emission TEM (FE-TEM, JEM-F200, JEOL), and UV-Vis absorption spectra were monitored by Cary 60 UV-Vis spectrophotometer (Agilent Technologies, USA).

### (2) Synthesis of Magnetic Nanoparticles and Plasmonic Magnetic Nanoparticles (PMNs)

0.34 g of FeCl₃·6H₂O, 0.6 g of sodium acetate and 0.11 g of trisodium citrate dehydrate were first dissolved in ethylene glycol (10 mL), the mixture was stirred and sonicated for 2 hours and then sealed in a Teflon-lined stainless-steel autoclave. The autoclave was heated at 200° C. and kept for 12 hours, and the magnetic products were collected with magnetic separation and washed with ethanol/deionized water (DIW) three times and dried at 60° C. before use. For PMNs, the same synthetic method as previously described [Fast and sensitive immuno-PCR assisted by plasmonic magnetic nanoparticles, Appl Mater Today, 23 (2021)] was used.

### (3) PPT-qiPCR Optical Device

Infrared-LED (850 nm peak wavelength, mounted on metal core PCB, 8.5 W power rating, 3.8 W radiant flux at 700 mA forward current, 12.4 V forward voltage, LZ4-40R608, LED Engine, CA, USA), Blue-LED (460 nm peak wavelength, mounted on metal core PCB, 10 W power rating, 3.9 W radiant flux at 700 mA forward current and 14 V forward voltage, LZ4-40B208, LED Engine, CA, USA), IR-thermometer (OPTCSTCLT15), Blue-LED (5 mm, 480 nm peak wavelength, 3.2 V forward voltage, 20 mA forward current, 4.1 cd, C503B-BCN-V0Z0461, CREE, Inc., NC, USA), FITC excitation filter (center wavelength=475 nm, bandwidth=35 nm, MF475-35, Thorlabs, Inc., NJ, USA), spectrophotometer (CCS200, Thorlabs, Inc., NJ, USA) and optical fiber (M0065-51-0034, Thorlabs, Inc., NJ, USA) were used to build PPT-qiPCR optical device. The LabVIEW program controlled power supply, thermocycling with LED, cooling fans, fluorescence measurement and temperature measurement.

### (4) Surface Carboxylation of PMNs (PMN-MPA)

Synthesis of magnetic nanoparticles and PMNs was performed as in a previous study (Fast and sensitive immuno-PCR assisted by plasmonic magnetic nanoparticles, Appl Mater Today, 23 (2021)). PMNs (0.25 mL, 1 mg·mL⁻¹ in DIW) and 3-Mercaptopropionic acid (0.5 μL, MPA) were mixed, then the mixture were sonicated for 30 minutes and centrifuged at 1,500 g for 8 minutes and washed with DIW, then finally redispersed in DIW (1 mg·mL⁻¹).

### (5) Manufacture of Plasmonic Immunomagnetic Nanoparticles (PIMNs)

The capture antibody was conjugated to PMNs via carbodiimide coupling. Specifically, PMN-MPA (20 μL, 1 mg·mL⁻¹ in DIW), N-(3-Dimethylaminopropyl)-N′-ethylcarbodimide hydrochloride (25 μL, 10 mg·mL⁻¹ in pH 5.5 MES buffer) and anti-human IL-6 Ab (10 μL, 0.5 mg·mL⁻¹ in pH 5.5 MES buffer) were mixed for 1 hour. The remaining active sites on the nanoparticles were blocked using bovine serum albumin (BSA, 2.0 wt %), and the product was washed with PBS (0.05 wt % Tween-20) in order to remove the excess capture antibodies and BSA, followed with resuspension in PBS, and stored at 4° C.

### (6) Immunoassay in PCR Tube

Functionalized nanoparticle-based immunoassay was conducted as follows: 25 μL IL-6 samples with various concentrations (1000, 500, 100, 10, 1, 0.1, 0.05, 0.025, 0 pg· mL⁻¹) were mixed with PIMNs (12.5 μL, 0.2 mg·mL⁻¹, 6 OD) and biotin anti-human IL-6 detection antibody (12.5 μL, 0.005 mg·mL⁻¹). After incubation for 1 hour on a shaker, it was washed three times with PBS (0.05 wt % Tween-20) using magnetic separation. The pre-conjugated streptavidin-dsDNA complex was obtained by mixing (in a ratio of 1:2) streptavidin (2.5 μL, 10 μg·mL⁻¹) and biotin-dsDNA (9.4 μL, 0.1 μM) for 30 minutes at room temperature. Then the pre-conjugated streptavidin-dsDNA complex was mixed for another 30 minutes. The formed sandwich immunocomplex with dsDNA template was washed with PBS (0.05 wt % Tween-20) for several times to reduce the background noise and prevent nonspecific interactions. Afterwards, the complex solution was redispersed in 5 μL PBS for further PCR reaction.

### (7) Preparation of PCR Mixtures for PPT-qiPCR

The 10 μL reaction mixture for PCR amplification was as follows: PCR buffer (1 μL, 10×), forward primer (0.5 μL, 10 μM), reverse primer (0.5 μL, 10 μM), dNTPs (0.2 μL, 10 μM), DNA polymerase (0.3 μL, 5 U·μL⁻¹), SYBR Green I (0.5 μL, 140× in DMSO), PIMN-sandwich (5 μL, 15 OD), nuclease free water (2 μL), and mineral oil (10 μL, on the mixture). PPT-qiPCR was conducted with two steps: an initial denaturation at 95° C. (5 seconds), 35 photothermocycles between 95° C. (1 seconds) and 72° C. (8 seconds), and the reaction was monitored by measuring the fluorescent emission signals at 530 nm in real-time during the annealing/extension at 72° C. for 1 second. After amplification, the product solution was mixed with TBE buffer (5 μL, 0.5×) and loading buffer (3 μL, 6×) and subjected to agarose gel (3%) electrophoresis analysis.

### (8) Colorimetric Assay

After PPT-qiPCR, MES buffer (9 μL, 0.1M, pH 4.7) and TMB (1 μL, 3 mM in final concentration) were added into 10 μL of the amplified PCR reaction mixture. Blue-Led was used to irradiate the resulting mixture for 10 minutes.

### 2. Experiment Result

### (1) Multifunctional Plasmonic Immunomagnetic Nanoparticles and Plasmonic Photothermocycle

### 1) Multifunctional Plasmonic Immunomagnetic Nanoparticles

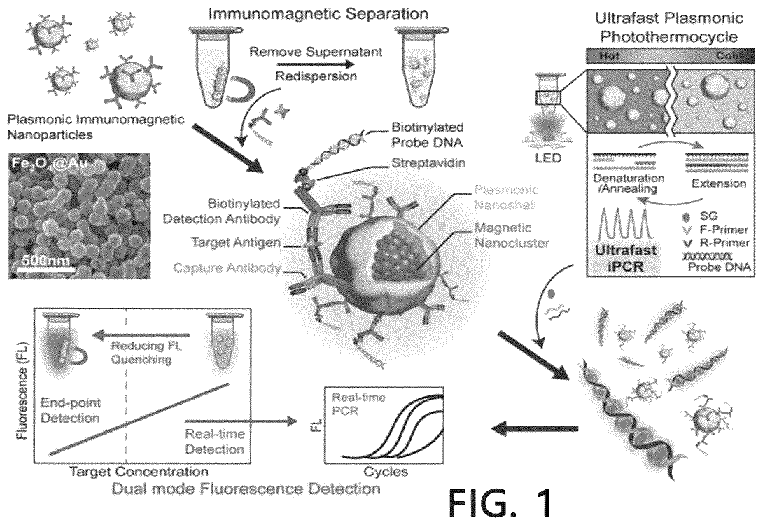

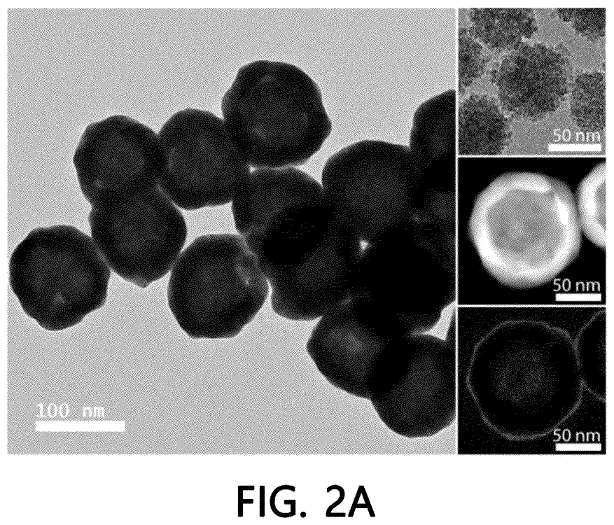

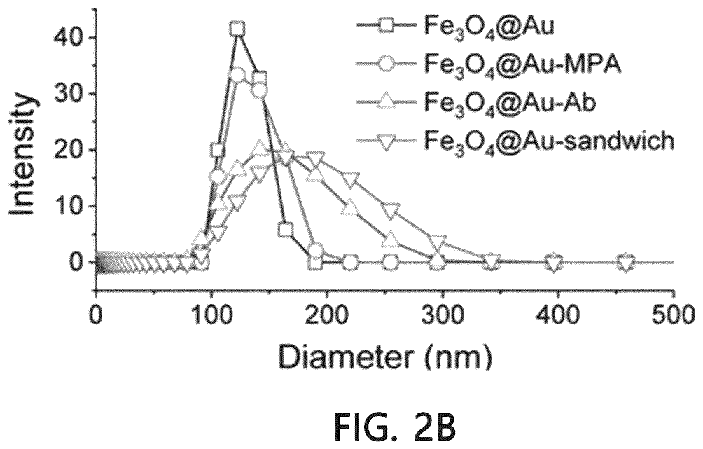

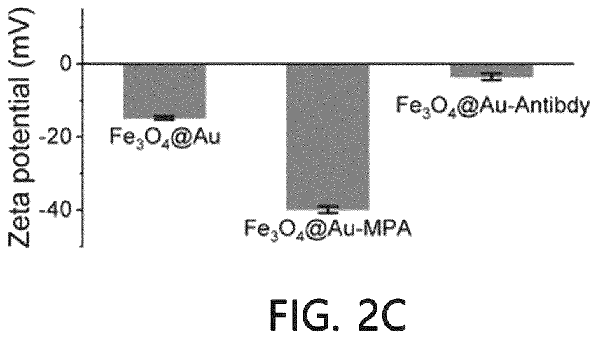

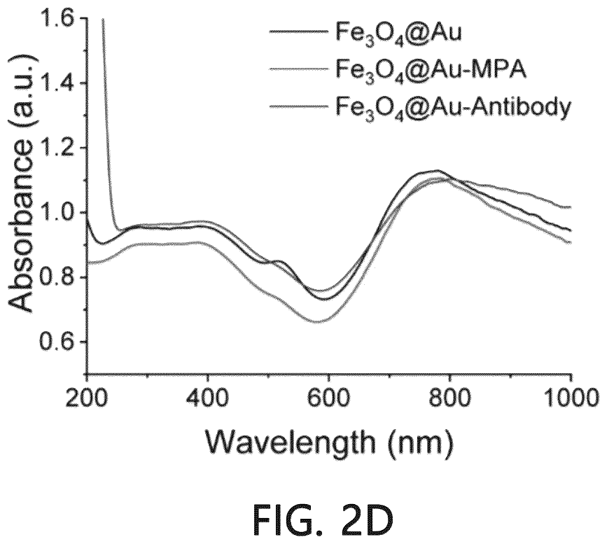

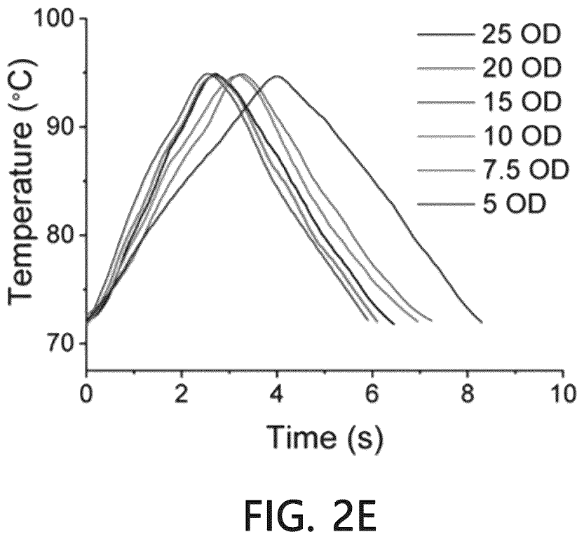

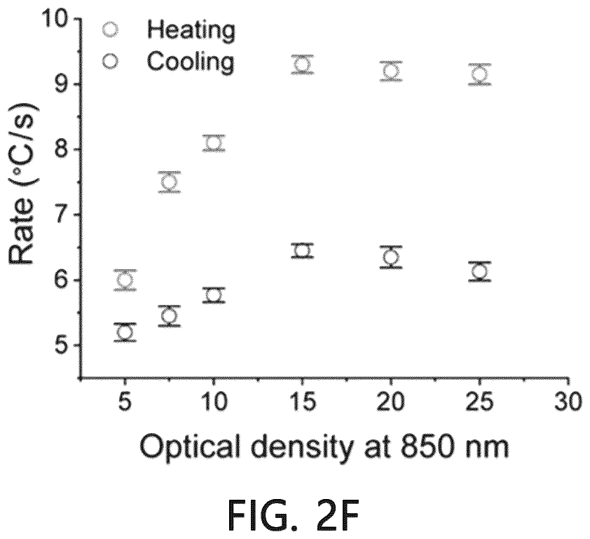

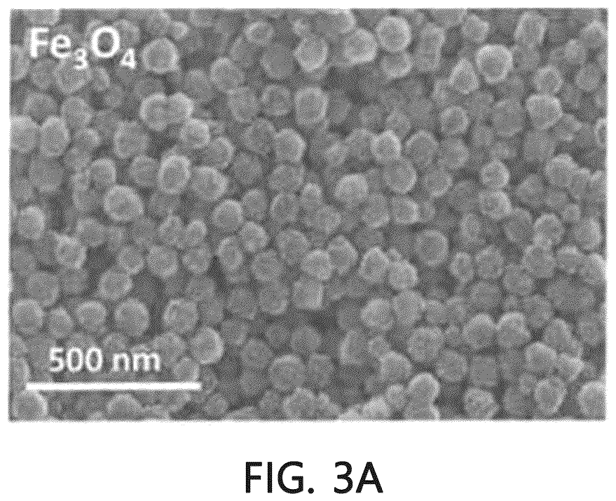

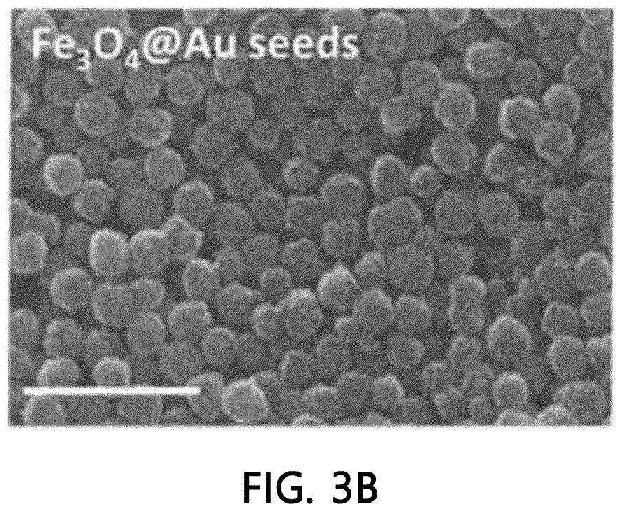

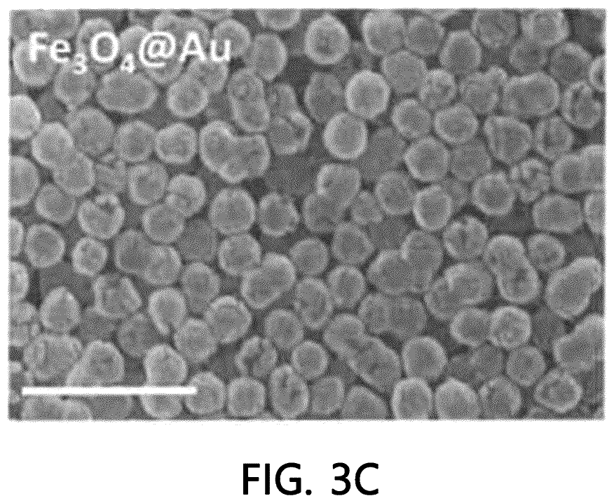

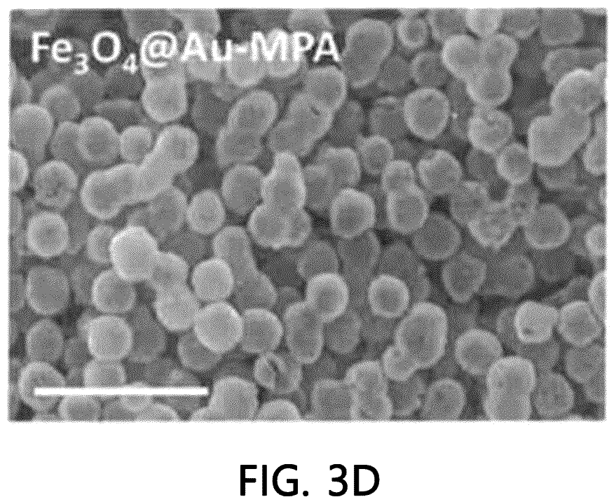

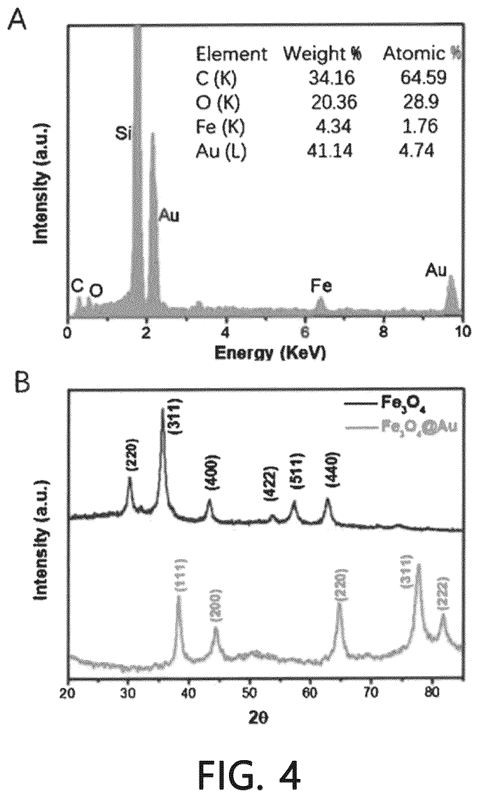

Multifunctional plasmonic immunomagnetic nanoparticles (PIMNs) are composed of Fe₃O₄ nanoclusters for magnetic properties, gold nanoshells for plasmonic photothermal conversion, and antibodies for immune response (FIG. 1). The magnetite Fe₃O₄ nanoclusters were prepared by a solvothermal reaction (FIGS. 2A and 3A). Subsequently, colloidal Au seeds were attached to Fe₃O₄ nanoclusters for Au shell layer growth (FIG. 3B). The attached Au seeds (Fe₃O₄@Au seeds) acts as nucleation sites to form a continuous gold shell, and the obtained plasmonic magnetic Fe₃O₄@Au nanoparticles were denoted as PMNs (FIGS. 2A and 3C). Afterwards, it was confirmed through electron microscope images that the magnetic core was completely covered with gold shell (FIG. 2A). X-ray powder diffraction (XRD) pattern showed the crystallinity of the magnetic core and Au shell and the different patterns between magnetic cores and PMNs further support the core-shell structure of PMNs (FIG. 4.) In addition, particle analysis with electron microscopy and dynamic light scattering confirmed that the PMNs were monodisperse with a uniform size of 108.7±10.4 nm (FIGS. 2B and 3C). For immobilizing capture antibody on the nanoparticle surface, bifunctional 3-Mercaptopropionic acid (MPA) containing sulfhydryl and carboxyl group was introduced as a linker between PMNs and antibodies. It was confirmed that PMNs were first modified with MPA through semi-covalent Au—S bonds and then antibodies were covalently conjugated on nanoparticle surface by carbodiimide coupling. The modification process was monitored by UV-Vis spectra, zeta potential and DLS analysis (FIGS. 2B to 2D). It was confirmed that the surface potential became more negative (−39.9 mV) after modification of MPA on PMNs, indicating modification of numerous carboxyl groups with negative charges. There was a significant shift of the ζ-potential in the positive direction after the modification of capture antibody, confirming the successful conjugation of antibodies on PMNs. The increase in hydrodynamic radius and LSPR peak broaden of PMNs further confirmed the successful antibody modification on the PMNs, and it was confirmed that the strong absorption of PMNs at IR wavelengths allows the IR light source to be used for photothermal conversion (FIG. 2D), preventing photobleaching of fluorescent dyes (SYBR© green, SG) resulting from prolonged light exposure.

### 2) Plasmonic Photothermocycle

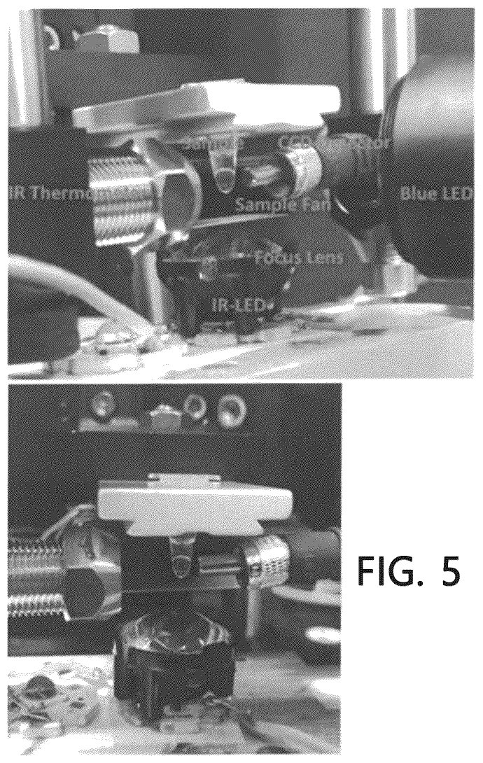

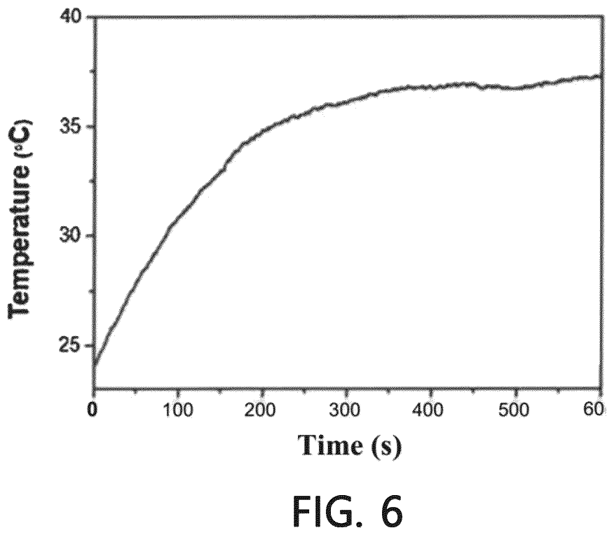

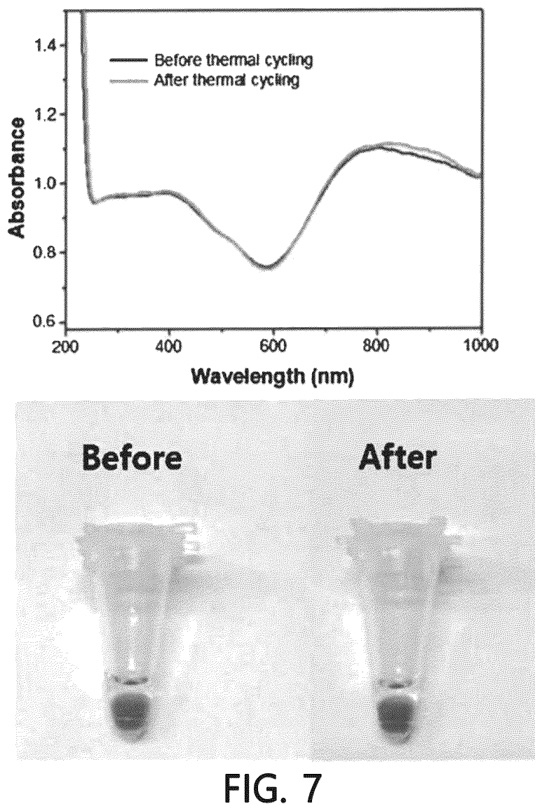

The thermocycling process, which is the core of iPCR, can be significantly shortened by utilizing the photothermal effect of plasmonic nanoparticles. A home-built IR-LED device was set up to study the photothermal conversion of PIMNs (FIG. 5). The solution temperature containing PIMNs was measured and recorded with a non-contact IR-thermometer. As shown in FIG. 2E, representative temperature profiles of one photothermocycle between 72° C. and 95° C. were identified with varying optical density (OD) of PIMNs. In addition, a linear rise in heating ramp rate was observed with increasing PIMNs OD from 5 to 15. It was confirmed that the shortest photothermocycling time of one cycle was 6 seconds for the 15 OD sample with heating rate at 9.32±0.13° C./s and cooling rate at 6.45±0.10° C./s (FIG. 2F). Given that the light energy absorbed by gold nanoparticles has no major energy loss channel other than the generation of heat, it is fair to expect a linear increase in heating rate. Temperature accuracy was 72.03±0.35° C. and 94.88±0.21° C. at 95° C. and 72° C., and it was confirmed that the heating rate was not significantly improved above 15.0 OD. It was confirmed that the above results were due to changes in light penetration depth or plasmonic resonance due to inter-particle coupling with increasing nanoparticle concentration. In addition, no obvious temperature changes were observed without nanoparticles under the IR-LED irradiation (FIG. 6), and it was confirmed that PIMNs exhibited good thermal stability after thermal cycling (FIG. 7).

### (2) Quantitative Immunoassay Using Real-Time and End-Point Fluorescence Dual-Readout

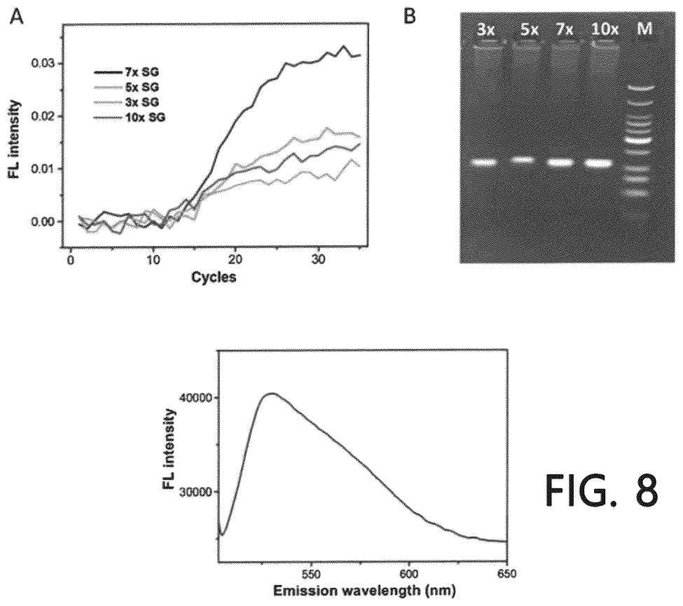

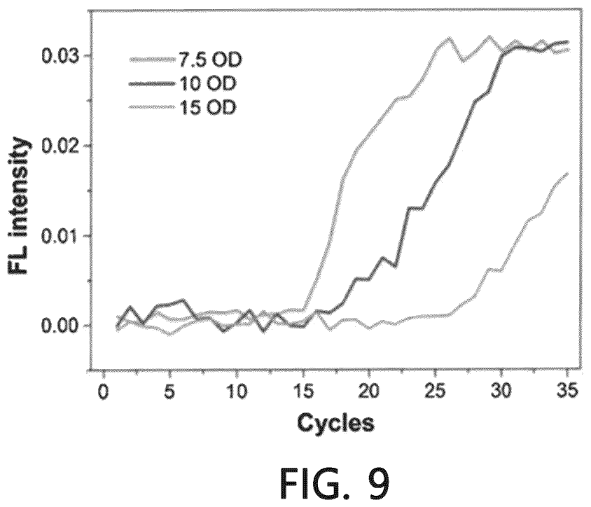

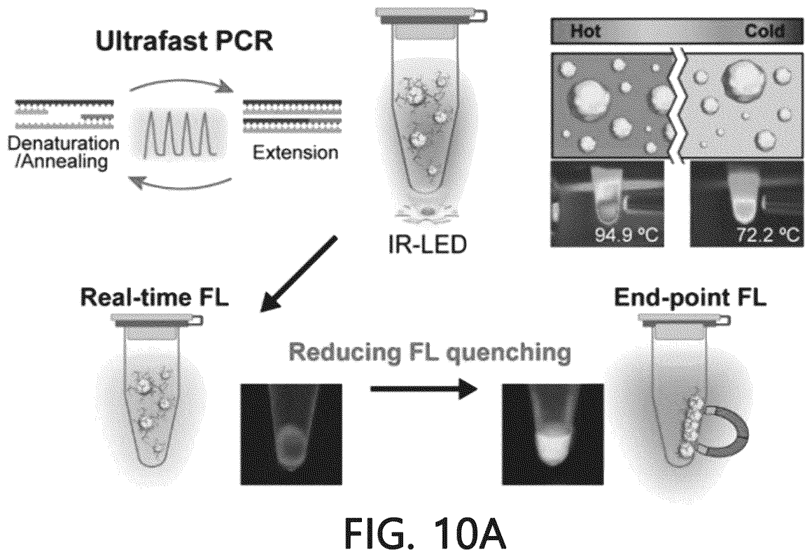

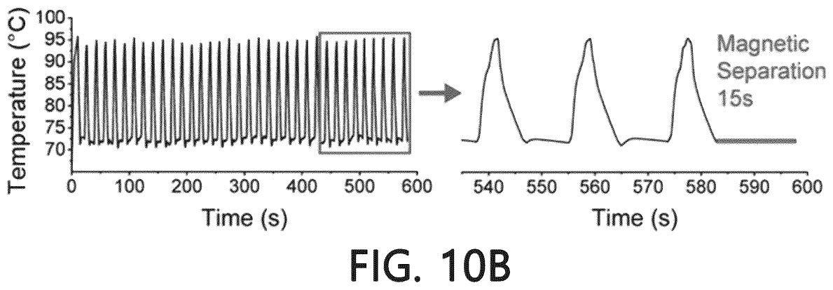

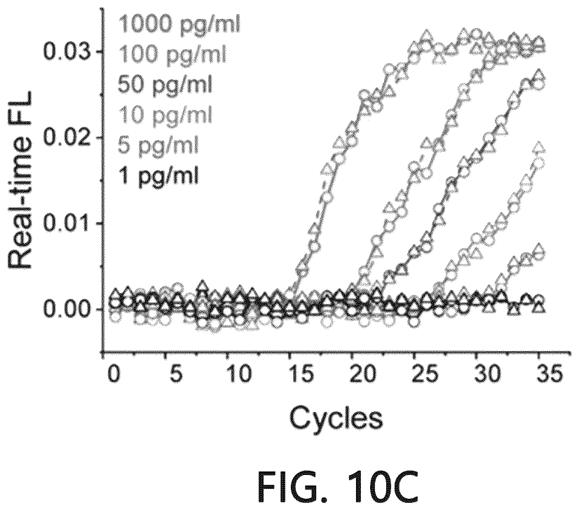

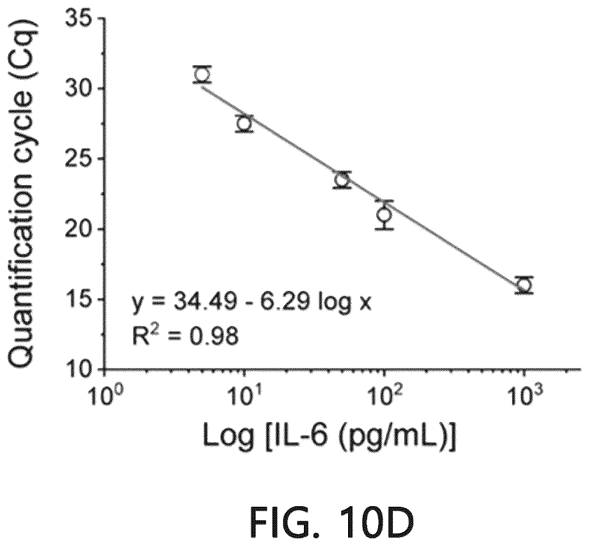

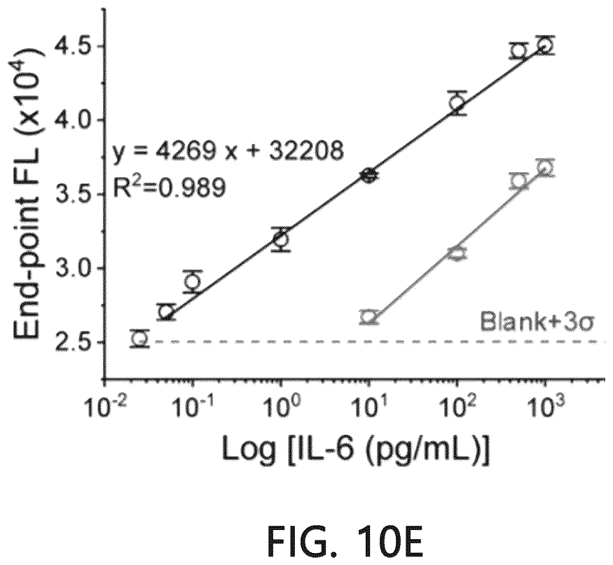

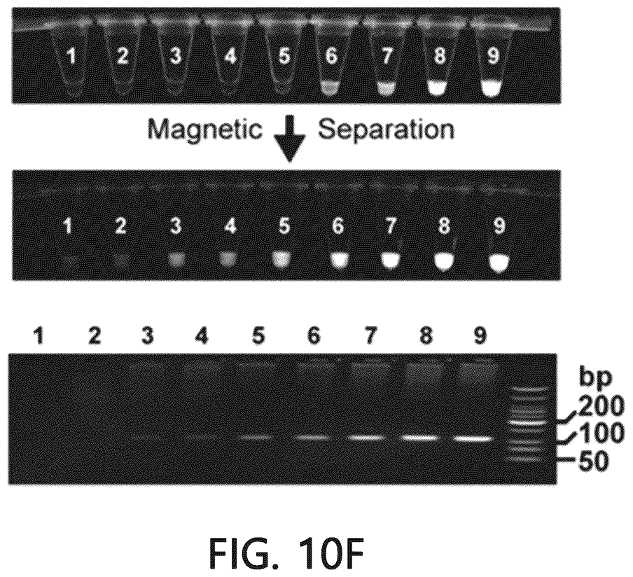

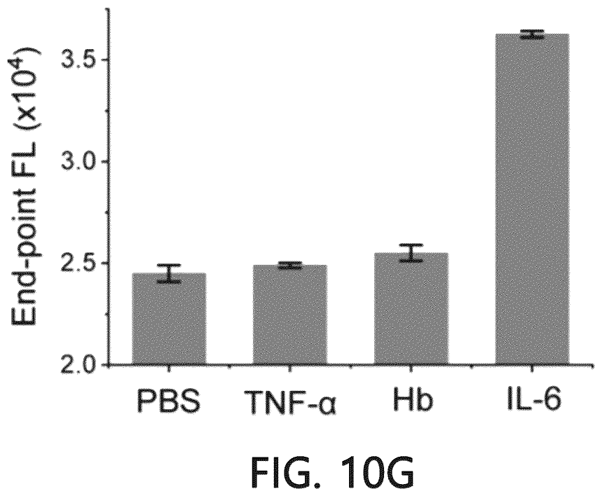

The monitoring capability of real-time PCR, which can quantify the amplified nucleic acid in real time, is being used as the gold standard in molecular diagnostics, and has been a benchmark technique in fluorescence-based point-of-care biosensor, multiplex PCR and immuno-PCR. Because the gold nanoparticles are known as efficient fluorescence quenchers, the concentration of PIMNs and SG dye were optimized to increase the fluorescence signal. Lambda DNA was amplified by a PPT-qiPCR using PIMNs, and the optimal conditions between PIMNs and SG dye concentrations were determined from amplification curves and gel electrophoresis assay results. As a result, it was confirmed that at fixed PIMNs concentration (7.5 OD), the fluorescence signal was proportional to the SG dye concentrations (×1 to ×7), however, further increasing the dye concentration to ×10 SG dye inhibited the amplification reaction (FIG. 8). Therefore, the SG dye concentration was fixed at ×7. On the other hand, the fastest heating rate was exhibited when the PIMNs concentration was 15.0 OD, but at this concentration, the degree of fluorescence quenching was higher than that of 7.5 OD (FIG. 9, FIG. 10C), so the concentration of nanoparticles was fixed at 7.5 OD later.

Next, a rapid and sensitive real-time PPT-qiPCR using PIMNs was used with varying concentrations of Human IL-6 under the optimized conditions (FIG. 10A). Anti-human IL-6 monoclonal capture antibody (Ab) labeled on PIMNs and biotinylated anti-human IL-6 monoclonal detection Ab were used to detect Human IL-6 by sandwich-type immunoassay. Serial dilutions of Human IL-6 were incubated with the capture antibody and detection antibody, after building sandwich structures and washing, biotinylated detection antibody was assembled with pre-conjugated streptavidin-DNA template to be used as a signaling probe in later PCR reaction. In addition, after washing three times, the PCR reaction mixtures including primers and polymerase were added to the PCR tube and ultrafast real-time PPT-qiPCR was performed using an IR-LED device. Temperatures of initial denaturation (5 seconds at 95° C.), and two-step 35 photothermocycles between denaturation (1 second, 95° C.) and annealing/extension (8 seconds, 72° C.) were controlled with non-contact IR thermometer. Afterwards, the fluorescence signal of each cycle indicating the amplification of the probe DNA was monitored in real-time with a blue-LED and spectrophotometer. It was confirmed that the amplification reaction was finished less than 10 minutes based on the temperature profiles (FIG. 10B). It was confirmed that compared to commercial thermal cyclers that transfer heat from the heating block to the reaction solution via PCR tubes, the system of the present invention with uniformly dispersed nanoparticles allows for much faster thermocycling as the heat can be directly and evenly distributed throughout the reaction mixture. A graph plotting the real-time amplification signal of PPT-qiPCR versus IL-6 concentration shows a typical qPCR amplification curve and was used to determine the quantification cycle (Cq) number (FIG. 10C). It was confirmed that Cq values showed a consistent linear relationship with IL-6 concentrations from 5 to 1000 pg·mL⁻¹ (FIG. 3D->FIG. 10D), and the achieved sensitivity and dynamic range of real-time PPT-qiPCR were compared with commercial ELISA kits and are shown in Table 1 below (Table 1).

TABLE 1

| | LOD | Dynamic Range |
|---|---|---|
| Company | (pg/mL) | (pg/mL) |
| Abeam | 2 | 6.25-200 |
| Thermo Fisher | <1 | 10.24-400 |
| BioLegend | 1.6 | 7.8-500 |
| R&D Systems | 0.7 | 3.1-300 |
| OriGene Technologies | <0.3 | 4.69-300 |
| Real-time PPT-qiPCR | | 5-1000 |
| Endpoint PPT-qiPCR | 0.0213 | 0.05-1000 |
| Colorimetric PPT-qiPCR | 0.0395 | 0.05-1000 |

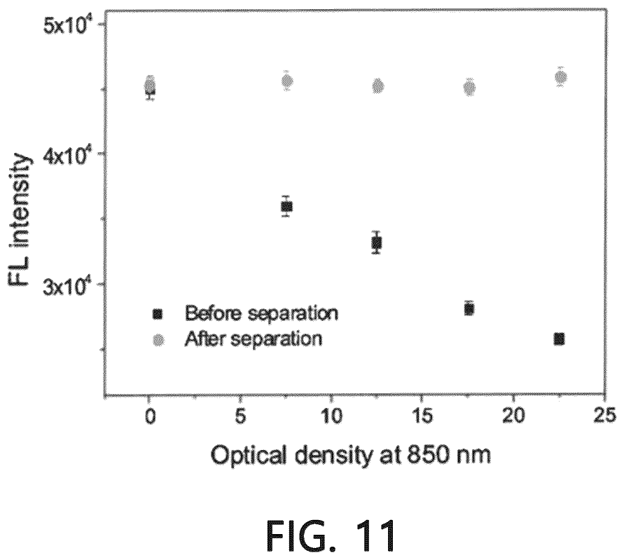

After demonstrating the successful application of real-time quantitative detection, the present inventors applied in-situ endpoint fluorescence measurements to the PPT-qiPCR system to achieve higher sensitivity. Simple magnetic separation can remove PIMNs from solution, thus greatly reducing fluorescence quenching. After the PPT-qiPCR reaction, PIMNs were collected at the bottom of the reaction tube with a magnet, and the in-situ endpoint fluorescence signal of the amplicons at 530 nm was measured and plotted against IL-6 concentrations (FIG. 10E). It was confirmed that the in-situ endpoint measurement including magnetic separation could be finished within 15 seconds (FIGS. 10B and 10E). As can be seen from the results, due to the elimination of fluorescence quenching, the in-situ end-point PPT-qiPCR was able to widen the detection range (0.05 to 1000 pg·mL⁻¹) and improve the sensitivity. And it was confirmed that the quantification plot also showed good linearity. It was confirmed that fluorescence images before and after magnetic separation, shown in FIG. 10F and nanoparticle concentration-dependent fluorescence signals shown in FIG. 11 directly supports that the decrease of fluorescence quenching is due to the elimination of nanoparticles. It was confirmed that the calculated analytical LOD from the correlation equation of [FL]=32208+4269 log [C] (R²=0.989) (FL: fluorescence signal of SG at 530 nm, C: concentration of IL-6) was 21.3 fg·mL⁻¹ (3 σ_{Blank}). In addition, as shown in Table 1 above, it was confirmed that the PPT-qiPCR shows about 100 times lower sensitivity compared to the LOD of commercial ELISA assay kits. It was confirmed that immunomagnetic process using PIMNs allows flexible sampling of the assay solution and shortening washing time, and reducing non-specific binding and background signal by allowing better thorough washing after antigen capture compared to commercial ELISA kits using microtiter plates. In addition, as a result of examining the selectivity of PPT-qiPCR for non-specific target proteins, as shown in FIG. 10G, it was confirmed that there was no obvious fluorescence signal distinct from the blank and non-specific targets, demonstrating good selectivity.

### (3) Colorimetric Detection

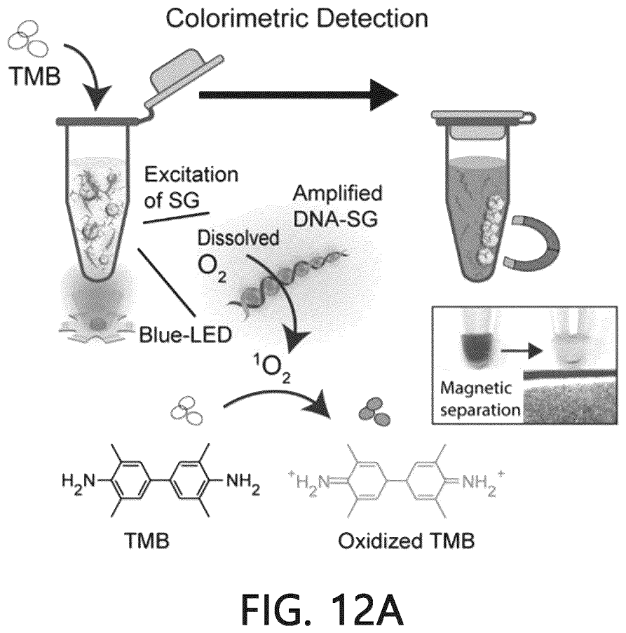

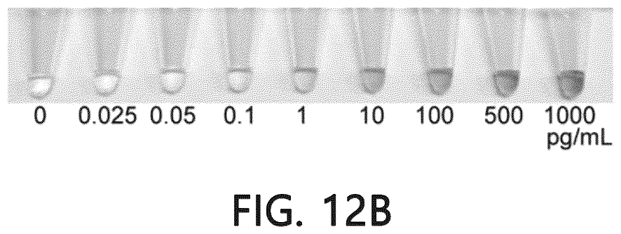

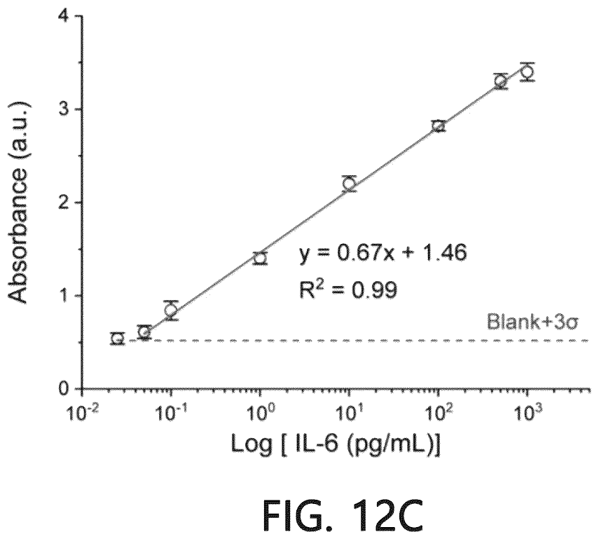

The colorimetric biosensor is one of the most suitable platform for POC diagnostics and resource-limited countries, because it is easy to operate and can eliminate expensive and complex instruments for signal detection. Maintaining the exponential amplification power of PCR, the present inventors further extended our system to colorimetric detection with chromogenic substrate that is typically used in ELISA (FIG. 12A). To demonstrate that the PPT-qiPCR method is applicable to a visually discernable colorimetric detection method, the photocatalytic properties of the DNA-SG complex to generate reactive oxygen species were utilized. Specifically, the oxidation of TMB can be induced by singlet oxygen generated in the DNA-SG complex when excited under a blue LED, and the generated singlet oxygen catalyzes the oxidation of TMB, causing color change, and the amount of singlet oxygen is directly related to the quantity of amplified target DNA.

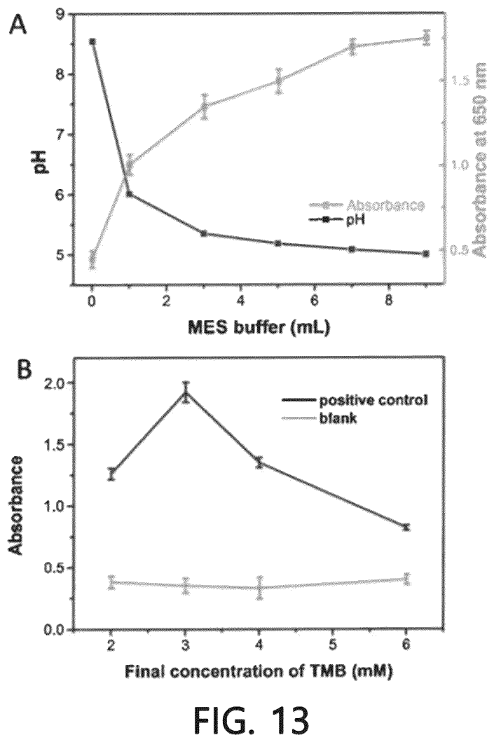

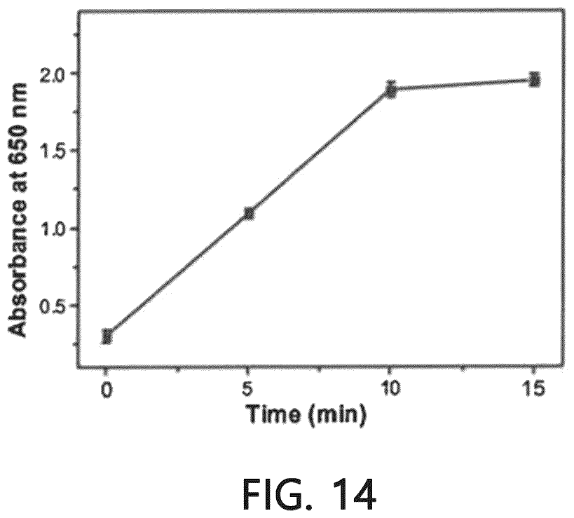

First, the photocatalytic activity of DNA-SG generating reactive oxygen species according to pH, TMB concentration and light irradiation time was investigated by fixing SG concentration at ×7. The absorbance intensity at 650 nm indicating TMB oxidation was monitored, and in order to adjust the pH of the solution, MES buffer (pH 4.7) was added after the PPT-qiPCR. As a result, it was confirmed that the optimum pH of TMB oxidation with DNA-SG was 5.0, which was compared with horseradish peroxidase (HRP) (FIG. 13A), and it was confirmed that the variation in absorbance according to the presence or absence of target DNA was greatest at 3 mM TMB concentration (FIG. 13B). When the light irradiation time was more than 10 minutes, there was no significant increase in absorbance (FIG. 14), and thus, further experiments were performed with pH and TMB concentrations at 5.0 and 3 mM respectively, with 10 minutes exposure time. After the immunomagnetic assay and PPT-qiPCR with serial dilutions of IL-6, the TMB solution in MES buffer was added to the PCR tube to check the colorimetric detection. Thereafter, after irradiation with blue-LED for 10 minutes, DNA-SG induced TMB oxidation, and the PMNs were then magnetically separated for 15 seconds to confirm a clear color change. Since the degree of photocatalytic activity of DNA-SG is directly correlated with the amount of the amplified amplicon, it was confirmed that the target can be quantified using the color change and saturation according to the amount of the amplified PCR product. As shown in FIG. 12B, the transition from colorless to blue served as a reliable indicator of the target's presence or absence, while the intensity of blue served as proof of the target concentration. A UV-Vis spectrophotometer was used to more accurately quantify color readings (FIG. 12C). The relationship between the absorbance at 650 nm and the IL-6 concentration was plotted, and it was confirmed that the curve showed a strong linear correlation with the equation [Abs]=1.46+0.67 log [C] (R²=0.99) (Abs: absorbance of TMB oxidation, C: concentration of IL-6). Similar to the fluorescence endpoint assay, the colorimetric method demonstrated a broad detection range from 0.05 pg·mL⁻¹ to 1 ng·mL⁻¹ and a LOD of 39.5 fg·mL⁻¹. Therefore, it was confirmed that even targets as low as 0.05 pg·mL⁻¹ are detectable with the naked eye. The amplified PPT-qiPCR product was further characterized by agarose gel electrophoresis (FIG. 10F), showing a clear change in band intensity with increasing IL-6 concentration.

What is claimed is:

1. A method for detecting target nucleic acids, comprising:
(a) binding capture antibody and plasmonic immunomagnetic nanoparticles (PIMNs);
(b) binding detection antibody and streptavidin-nucleic acid complex;
(c) mixing the nanoparticles bound to the capture antibody and the complex bound to the detection antibody;
(d) separating the mixed nanoparticles and complex;
(e) amplifying target nucleic acids using the complex, primer, and nucleic acid polymerase; and
(f) detecting target nucleic acids.

2. The method according to claim 1, wherein in step (a), the plasmonic immunomagnetic nanoparticles have magnetism and photothermal properties.

3. The method according to claim 1, wherein in step (a), the capture antibody is covalently bound to the surface of the nanoparticle.

4. The method according to claim 1, wherein in step (b), the detection antibody is biotinylated.

5. The method according to claim 1, wherein in step (c), the nanoparticles and the complex are mixed in a sandwich structure.

6. The method according to claim 1, wherein in step (e), the polymerase is at least one selected from the group consisting of Taq polymerase, VENT polymerase, DEEPVENT polymerase, PWO polymerase, and Pfu polymerase.

7. The method according to claim 1, wherein in step (e), the amplification is performed through photothermal cyclic amplification.

8. The method according to claim 1, wherein in step (f), the detection is performed through at least one selected from the group consisting of colorimetric assay, fluorescence assay, Raman assay, and gel electrophoresis.

9. Multifunctional plasmonic immunomagnetic nanoparticles, comprising iron oxide nanoclusters, gold nanoshells and antibodies.

10. The nanoparticles of claim 9, wherein the iron oxide nanoclusters have magnetism.

11. The nanoparticles of claim 9, wherein the gold nanoshells have photothermal properties.


SEQUENCE LISTING (2 sequences)

SEQ ID NO: 1 - DNA, 28 bp, synthetic construct
  source 1..28: mol_type = other DNA
catcgtctgcctgtcatgggctgttaat

SEQ ID NO: 2 - DNA, 26 bp, synthetic construct
  source 1..26: mol_type = other DNA
tcgccagcttcagttctctggcattt